(19) United States
(12) Patent Application Publication
(10) Pub. No.: US 2026/0363686 A1
(43) Pub. Date: Nov. 19, 2026

(54) LIGATING DEVICE

(71) Applicant: BOSTON SCIENTIFIC MEDICAL DEVICE LIMITED, Galway (IE)

(72) Inventors: Sushil KUMAR, Muzaffarpur (IN); Amit KUMAR, Noida (IN); Neeraj KUMAR GARG, Gurgaon (IN); Swami UPADHYAY, Raipur (IN)

(21) Appl. No.: 19/649,930

(22) Filed: Apr. 16, 2026

Related U.S. Application Data
(60) Provisional application No. 63/805,591, filed on May 14, 2025.

(51) Int. Cl. A61B 17/00; A61B 90/00

(52) U.S. Cl. CPC A61B 17/00234; A61B 90/03; A61B 2017/00238; A61B 2017/0034; A61B 2017/00367; A61B 2017/00407; A61B 2017/00867; A61B 2090/034

(57) ABSTRACT

A system includes a ligating device and a delivery device with a catheter extending longitudinally from a proximal end to a distal end and defines a lumen. The ligating device is slidably received within the lumen and includes a proximal portion and a distal portion extending along a predefined curve shape. The ligating device moves from an insertion configuration, in which the ligating device is housed within the lumen such that the ligating device is held in a straightened configuration, to an extended configuration, in which the ligating device is moved distally out of the catheter so that the distal portion reverts toward the shape to extend about a target tissue. The proximal portion includes, at a proximal end, a grommet with a channel extending therethrough so that when a distal end of the ligating device is drawn proximally through the channel, the ligating device tightens about the tissue.

### PRIORITY CLAIM

The present disclosure claims priority to U.S. Provisional Patent Application Ser. No. 63/805,591 filed May 14, 2025; the disclosure of which is incorporated herewith by reference

### FIELD

The present disclosure relates to endoscopic devices and, in particular, relates to endoscopic ligature devices for treating tissue along the gastrointestinal tract.

### BACKGROUND

Ligating loop devices are often used in endoscopic procedures such as, for example, polypectomies and mucosal resections. Ligating loop devices generally comprise a suture formed in a ligature loop with a knot or other mechanism configured to tighten the loop about a tissue. In use, the ligating loop device may be inserted to a target area within, for example, the gastrointestinal tract to ligate and close off vessels or tissues to prevent bleeding after the removal of a polyp or to address other issues. During a polypectomy procedure, for example, the ligating loop device is positioned about a base of a pedicle of a polyp and tightened around the pedicle. A polypectomy—i.e., removal of a polyp—may then be performed by resecting a portion of the tissue above the deployed ligating loop. Thus, precise positioning of the ligating loop is required for successful treatment.

Current ligating loop devices generally include a closed loop that must be maneuvered over the polyp and moved downward over the polyp until the loop encircles the pedicle of the polyp. In some cases, it may be challenging to position the ligating loop device about the target portion of a pedicle/tissue as the ligating loop is floppy and may be difficult to maneuver in some areas. In addition, other challenges such as, for example, loop slippage, loop transection and/or deployment issues may result in failure of the device.

### SUMMARY

The present disclosure relates to a system for ligating tissue. The system includes a delivery device including a catheter extending longitudinally from a proximal end to a distal end and defining a lumen extending therethrough. In addition, the system includes a ligating device slidably received within the lumen of the catheter and including a proximal portion configured as a fastener and a distal portion extending along a predefined curve shape, the ligating device configured to be moved from an insertion configuration, in which the ligating device is housed within the lumen of the catheter such that the ligating device is held in a straightened configuration, to an extended configuration, in which the ligating device is moved distally out of the catheter so that the distal portion reverts toward the predefined curve shape to extend about a target tissue, the proximal portion including a grommet at a proximal end thereof, the grommet including a channel extending therethrough so that when a distal end of the ligating device is drawn proximally through the channel of the grommet, the ligating device is configured to be tightened about the target tissue.

In an embodiment, the system further includes a control member slidably received within the catheter and extending longitudinally from a proximal end to a distal end including a grasper configured to grasp the distal end of the ligating device, the control member including a shoulder and a distal portion extending distally therefrom to be received within the channel of the grommet, the shoulder being sized, shaped and configured to abut a portion of the ligating device such that a distal movement of the control member relative to the catheter correspondingly moves the ligating device distally relative to the catheter.

In an embodiment, the grasper is configured as a hook.

In an embodiment, the distal end of the ligating device includes a loop configured to be grasped via the grasper of the control member.

In an embodiment, the proximal portion is defined via a proximal section including the grommet and a distal section including engaging elements, each of the engaging elements configured to engage a portion of the grommet as the engaging elements are drawn proximally through the channel of the grommet to tighten the ligating device about the target tissue.

In an embodiment, the engaging element and the channel of the grommet engage one another via a ratchet mechanism.

In an embodiment, the proximal section and the distal section are connected to one another via a midsection, the midsection extending distally from a wall of the grommet so that a longitudinal axis of the midsection is offset from a longitudinal axis of the channel of the grommet.

In an embodiment, the distal portion of the ligating device is formed of a Nitinol wire.

In an embodiment, the proximal portion of the ligating device is formed of a polymer material.

In an embodiment, the delivery device further includes a handle assembly connected to the proximal end of the catheter and to the proximal end of the control member, the handle assembly including a first actuator for controlling a longitudinal movement of the control member relative to the catheter and a second actuator for controlling an axial motion of the control member relative to the catheter.

In addition, the present disclosure relates to a ligating system. The system includes a catheter extending from a proximal end to a distal end and including a lumen extending therethrough; a ligating device extending along a longitudinal axis from a proximal end including a grommet to a distal end, a distal portion of the ligating device formed of a wire extending along a predefined curve shape, the ligating device configured to be moved from an insertion configuration, in which the ligating device is housed within the lumen of the catheter such that the distal portion is held in a straightened configuration, to an extended configuration, in which the ligating device is moved distally out of the catheter so that the distal portion reverts toward the predefined curve shape to extend about a target tissue; and a control member slidably received within the lumen of the catheter and extending longitudinally from a proximal end to a distal end including a grasper, the control member including a shoulder and a distal portion extending therefrom, the shoulder configured to abut a portion of the ligating device so that a longitudinal movement of the control member relative to the catheter correspondingly moves the ligating device longitudinally relative to the catheter, the distal portion of the control member received within a channel of the grommet so that the grasper extends distally therefrom to grasp the distal end of the ligating device and draw the distal end of the ligating device proximally through the channel of the grommet to tighten the ligating device about the tighten the ligating device about the target tissue.

In an embodiment, the distal end of the ligating device includes a loop configured to be engaged via a hook of the grasper.

In an embodiment, the ligating device includes a proximal portion from which the distal portion extends, the proximal portion defined via a proximal section including the grommet and a distal section including engaging elements, each of the engaging elements configured to engage a portion of the grommet via a ratchet mechanism as the engaging elements are drawn proximally through the channel of the grommet to tighten and fix the ligating device about the target tissue.

In an embodiment, the proximal section and the distal section are connected to one another via a midsection, the midsection extending distally from a wall of the grommet so that a longitudinal axis of the midsection is offset from a longitudinal axis of the channel of the grommet.

In an embodiment, the distal portion of the ligating device is formed of a Nitinol wire and the proximal portion of the ligating device is formed of a polymer material.

In addition, the present disclosure relates to a method for ligating tissue. The method includes advancing a ligating device to a target area within a patient body, in an insertion configuration in which the ligating device is housed within a catheter of a delivery device; moving the ligating device from the insertion configuration toward an extended configuration in which the ligating device is moved distally out of the catheter so that a distal portion thereof reverts toward a predefined curve shape to extend about a target tissue; and drawing a distal end of the ligating device through a channel of a grommet at a proximal end of the ligating device to tighten a loop of the ligating device formed about the target tissue.

In an embodiment, the method further includes drawing the distal end of the ligating device further proximally relative to the grommet until a desired level of tightness is achieved and engaging structures along the ligating device engage a portion of the grommet to hold the ligating device in the desired tightened configuration to ligate the target tissue.

In an embodiment, the method further includes rotating the ligating device relative to the catheter to position the ligating device in a desired orientation relative to the target tissue so that the target tissue extends about the target tissue as it is moved toward the extended configuration.

In an embodiment, the ligating device is moved from the insertion configuration toward the extending configuration via a control member slidably received within the catheter, the control member including a shoulder sized, shaped and configured to abut a portion of the ligating device such that a distal movement of the control member relative to the catheter correspondingly moves the ligating device distally relative to the catheter.

In an embodiment, a grasper at a distal end of the control member grasps the distal end of the ligating device to draw the distal end proximally through the channel of the grommet.

### BRIEF DESCRIPTION

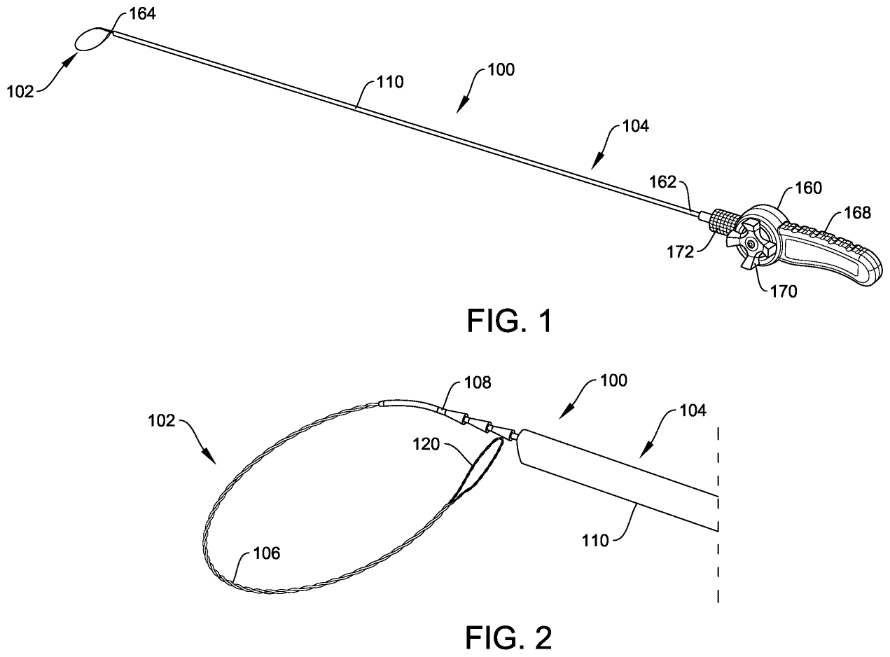

FIG. 1 shows a perspective view of a system according to an exemplary embodiment of the present disclosure;

FIG. 2 shows a perspective view of a distal portion of the exemplary system according to FIG. 1;

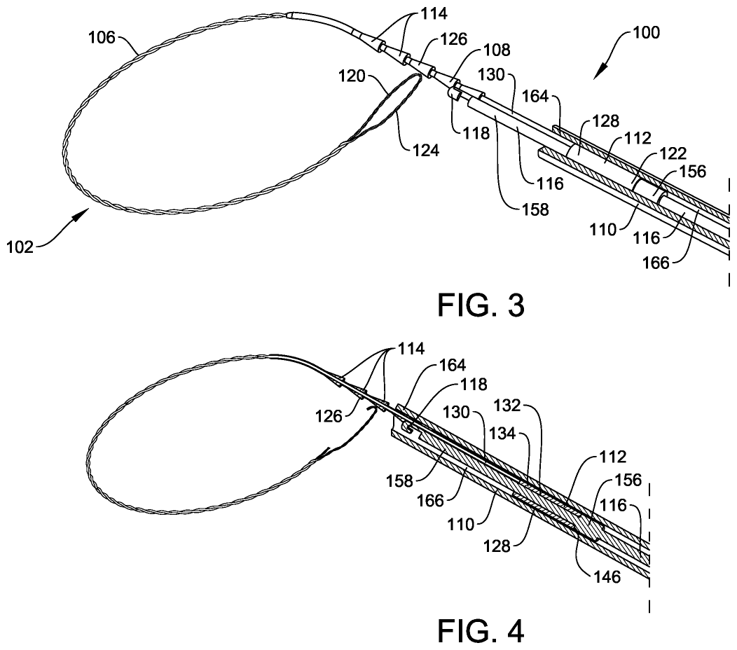

FIG. 3 shows a partially cross-sectional perspective view of the distal portion of the exemplary system according to FIG. 1;

FIG. 4 shows a cross-sectional side view of a distal portion of the exemplary system according to FIG. 1;

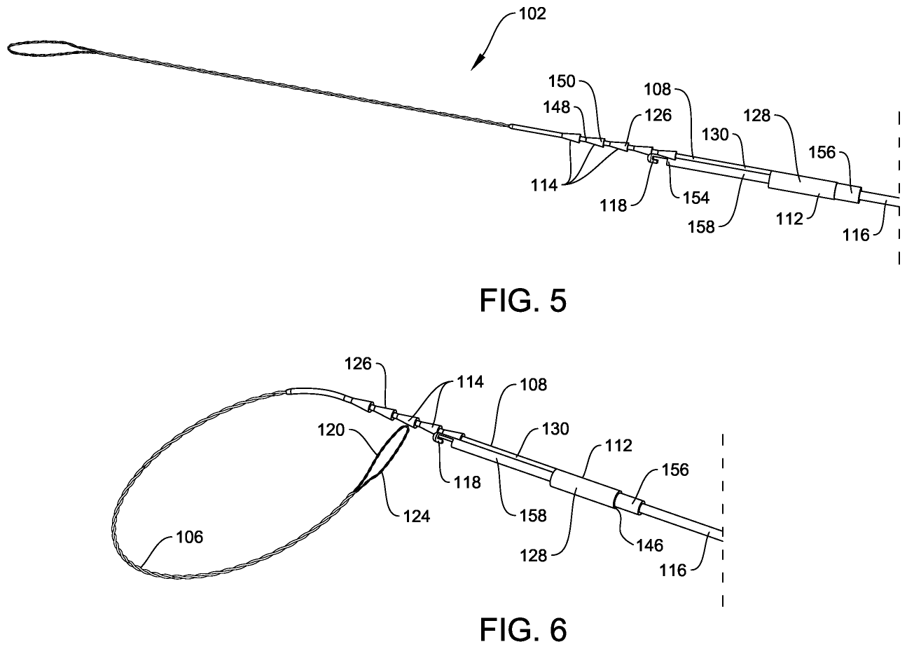

FIG. 5 shows a side view of a ligating device and a portion of a control member, in a straightened configuration, according to the exemplary system of FIG. 1;

FIG. 6 shows a side view of the ligating device and a portion of the control member, in a curved configuration, according to the exemplary system of FIG. 1;

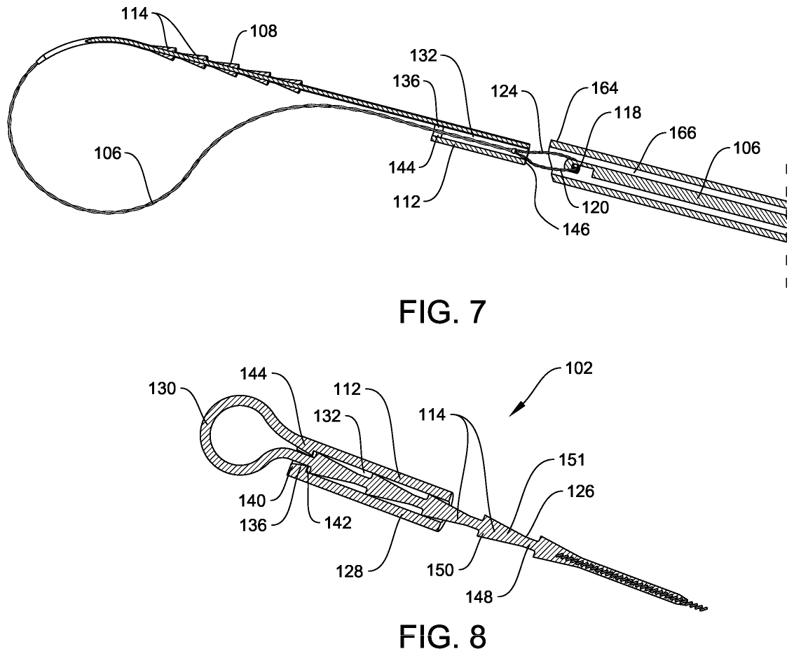

FIG. 7 shows a cross-sectional side view of the exemplary system according to FIG. 1, during a tightening of the ligating device; and

FIG. 8 shows a cross-sectional side view of the ligating device in a finally deployed/tightened configuration according to the exemplary system of FIG. 1.

### DETAILED DESCRIPTION

The present disclosure may be further understood with reference to the following description and the appended drawings, wherein like elements are referred to with the same reference numerals. The present disclosure relates to an endoscopic device and, in particular, an endoscopic ligating device. Exemplary embodiments of the present disclosure describe a system comprising a ligating device configured to be positioned around a target portion of tissue to be ligated (e.g., pedicle of a target polyp) via a delivery device. According to an exemplary embodiment, the ligating device includes a proximal proportion formed of a polymer material and a distal portion formed of a wire having a predefined shape that extends along a curve. The proximal portion comprises a proximal section including a grommet and a distal section including engaging structures configured to engage a portion of a channel of the grommet. In use, the ligating device is moved distally out of a catheter freeing the distal portion to extend along its predefined shape. This shape is then looped about and/or positioned to encircle the target tissue.

Once the target tissue has been encircled as desired, a grasper or other suitable tool is used to grasp a distal-most end of the distal portion and draw it proximally through the grommet to tighten the looped ligating device about the target tissue. As the distal-most end of the distal portion is moved further proximally relative to the grommet, engaging structures along the distal section of the proximal portion engage the grommet to maintain the ligating device in a tightened configuration. The distal-most end may be drawn proximally relative to the grommet until the ligating has been tightened about the target tissue to the desired degree.

Once tightened as desired, the grasper releases the distal-most end so that the insertion device may be withdrawn from the body, leaving the ligating device tightened about the target tissue. It will be understood by those of skill in the art that the terms “proximal” and “distal,” as used herein, are intended to refer to a direction toward and away from, respectively, a user of the device. Furthermore, it will be understood that, although the exemplary ligating device is described with respect to polypectomies, the ligating device of the present disclosure may be utilized in any of a variety of procedures in which a looped ligation of a target tissue may be desired.

As shown in FIGS. 1-8, a system 100 according to an exemplary embodiment of the present disclosure comprises a ligating device 102 configured to be positioned about a target portion of tissue (e.g., pedicle of a polyp) via a delivery device 104. The ligating device 102 includes a distal portion 106 formed of a wire formed so that, in an unstressed state, it extends along a predefined curved and a proximal portion 108 configured to tighten and fasten the ligating device 102 about the target tissue. In an exemplary embodiment, the proximal portion 108 includes a series of engaging elements 114 formed along a section of the proximal portion 108 immediately proximal of the distal portion 106 and a grommet 112 at a proximal end of the proximal portion 108. According to an exemplary embodiment, the ligating device 102 is moved from an insertion configuration, in which the ligating device 102 is housed within a catheter 110 of the delivery device 104 holding the distal portion 106 in a substantially straightened configuration, to an extended configuration, in which the distal portion 106 extends distally out of the catheter 110 so that the distal portion 106 reverts toward the predefined curved shape which facilitates the looping of the distal portion 106 about or encircling the target tissue.

Once the ligating device 102 has been looped about the target tissue, the user extends a grasper 118 at a distal end of a control member 116 to grasp a distal end 120 of the ligating device 102 so that the distal end 120 may be drawn proximally through the grommet 112 to close and tighten the loop about the target tissue. The distal end 120 is drawn proximally relative to the grommet 112 until the proximal portion 108 enters the grommet 112. The user continues to draw the distal end 120 proximally relative to the grommet 112 until one of more of the engaging elements 114 of the proximal portion 108 engages the grommet 112. As the engaging elements 114 enter the grommet 112, as will be described in more detail below, the engaging elements 114 pass a shoulder of the grommet 112 which abuts a proximal shoulder of the engaging element 114 locking the proximal portion 108 against movement distally out of the grommet 112 maintaining the loop in a desired tightened configuration. Once a desired degree of tightening has been achieved (e.g., a desired number of engaging elements 114 have been drawn into the grommet 112), the user releases the distal end 120 from the grasper 118 so that the delivery device 104 separating the ligating device 102 from the catheter 110 so that the ligating device 102 may be left in place around the target tissue ligating this tissue while the catheter 110 is removed from the body.

The ligating device 102 extends longitudinally from a proximal end 122 to the distal end 120 and comprises the distal portion 106 and the proximal portion 108. As described above, the distal portion 106 is movable from the insertion configuration (straightened within the catheter 110 or bending along any curve the catheter 110 assumes as it traverses a tortuous path to the target site within the body) to the extended configuration in which the distal portion 106 extends out of the catheter 110 to return to its curved shape to the extent permitted by surrounding tissue, etc. For illustrative purposes, FIG. 5 shows the ligating device 102 as it would appear in the straightened, insertion configuration. As the ligating device 102 is moved distally out of the catheter 110, the distal portion 106 returns to its predefined curved shape, as shown in FIG. 6. As would be understood by those skilled in the art, a length of the distal portion 106 and/or a curvature of the distal portion 106 may be selected based on a size and/or shape of the target tissue or other considerations relevant to a particular procedure. The distal portion 106 along with a portion of the proximal portion 108 may fully encircle the target tissue, as will be described in further detail below.

In an exemplary embodiment, the distal portion 106 is formed of a Nitinol wire which, as would be understood by those of skill in the art, has superelastic and shape memory properties. In one example, the distal portion 106 is flexible but is biased toward the predefined curved shape so that, the distal portion 106 is constrained (e.g., elastically deformed) and held in the straightened configuration by an interior surface of the catheter 110 in the insertion configuration. As the ligating device 102 is moved toward the extended configuration (i.e., as the distal portion 106 is extended distally out of the distal end of the catheter 110, the distal portion 106 is freed from the constraint of the catheter 110 and permitted to revert toward the curved shape under its natural bias.

In another example, the distal portion 106 is deformed toward the straightened configuration when housed in the catheter 110 in the insertion configuration. As the distal portion 106 is extended out of the distal end of the catheter 110 (i.e., moved toward the extended configuration), a temperature of the living body within which the ligating device 102 is delivered causes the distal portion 106 to revert toward a memorized curved shape. That is, in this embodiment, the distal portion 106 is not biased toward the predetermined curved shape. Rather, the wire of the distal portion 106 is formed of a shape memory material (e.g., Nitinol) that has been treated so that the predetermined curved shape is “memorized” as would be understood by those skilled in the art. Although the distal portion 106 is specifically described as formed of Nitinol, it will be understood by those of skill in the art that the distal portion may be formed of any of a variety of materials having superelastic and/or shape memory properties which allow movement of the ligating device 102 and, in particular the distal portion 106, from the substantially straight insertion configuration toward the curved extended configuration described above either through the memorization of a shape in a shape memory material or through the imparting of a bias toward the desired shape in any conventional manner as would be understood by those skilled in the art.

In an exemplary embodiment, the distal portion 106 is formed by twisting a single piece of wire so that the distal end 120 is plastically deformed to include a loop 124. The distal portion 106 of the exemplary embodiment may be stiffer than conventional ligating loop devices. In one embodiment, the distal portion 106 is formed of a 0.004 inch gauge wire. The size of the loop 124, as will be described in further detail below, is selected to facilitate grasping of the distal end 120 during a tightening of the ligating device 102 about the target tissue. It will be understood by those of skill in the art, however, that the distal portion 106 and/or the distal end 120 may have any of a variety of configurations so long as the distal portion 106 facilitates looping of the ligating device 102 about the target tissue and the distal end 120 is configured to be grasped by the grasper 118, as will be described in further detail below.

According to an exemplary embodiment, the proximal portion 108 is formed of a polymer material defined via three sections—a distal section 126 including the engaging elements 114, a proximal section 128 including the grommet 112, and a midsection 130 connecting the distal section 126 and the proximal section 128. As described above, the proximal portion 108 acts as a fastener and is configured to facilitate tightening of the ligating device 102 and maintaining the ligating device 102 in this tightened configuration. In particular, the engaging elements 114 along the distal section 126 is configured to engage the grommet 112 as the distal section 126 is drawn proximally into the distal end of the grommet 112 with a ratcheting action that successively locks each of the engaging elements as it enters the grommet 112 so that the engaging elements 114 are prevented from moving out of the distal end of the grommet 112.

The grommet 112 extends longitudinally and in one embodiment is configured as a substantially tubular element including a channel 132 extending therethrough. The channel 132 includes a pawl 136 or other structure configured to engage the engaging elements 114 as they enter the distal end of the grommet 112. In an exemplary embodiment, the engaging elements are configured with a diameter that gradually increases from a distal end to a maximum diameter at the proximal end of each engaging element. The proximal end of each of the engaging elements 114 is formed as a substantially planar disc substantially perpendicular to a longitudinal axis of the distal portion 106 or otherwise forms a shoulder. Thus, as the distal portion 106 is drawn into the distal end of the grommet 112, the small diameter distal end of each engaging element 114 engages the pawl 136.

As the engaging element 114 is drawn proximally into the grommet 112, the pawl 136 slides along the tapered surface of the engaging element 114 until it reaches the proximal end of the engaging element 114. At this point, the pawl 136 snaps radially inward so that contact with the planar proximal end of the engaging element 114 prevents the engaging element 114 from moving back out of the distal end of the grommet 112. Thus, as each of the engaging elements 114 is moved proximally past the pawl 136 into the grommet 112, the ligating device 102 is progressively tightened and is maintained in the tightened configuration by the interaction of the pawl 136 with the proximal end of the last engaging element that has been drawn into the grommet 112.

In an exemplary embodiment, the pawl 136 is configured as a tab (see FIG. 8) extending radially inward within the grommet 112 from a first end 140 connected to a surface of the channel 132 to a second end 142, which is biased to extend toward a centerline of the channel 132 in a first position. The pawl 136 is configured to be flexed (e.g., elastically deformed) away from the biased first position to a second position, in which the second end 142 is flexed so that the second end 142 is proximal of the first end 140. This permits the engaging elements 114 to be moved proximally therepast. The pawl 136 is moved from the first position toward the second position as by contact with the tapered surface of each of the engaging elements 114 as it is moved proximally therepast. Once the engaging element 114 has been moved proximally so that its proximal end is proximal of the second end 142 of the pawl 136, the pawl 136 returns to the first position so that it lockingly engages the proximal end of this engaging element 114 so that the engaging element 114 cannot be moved distally past the pawl 136 out of the grommet 112.

Although exemplary embodiments show and describe the pawl 136 configured as a tab extending from the surface of the channel 132 of the grommet 112, it will be understood by those of skill in the art that the pawl 136 may have any of a variety of configurations so long as the pawl 136 is configured to move between the first and second positions, as described herein, to permit proximal passage of each of the engaging elements 114 therepast while preventing the engaging elements 114 from moving distally out of the grommet 112. In another embodiment, for example, the pawl 136 may be configured as a lever pivotally coupled to the surface of the channel 132 so that the lever I movable between the first and second position by contact with the tapered surfaces of the engaging elements 114 but is not movable from the first position by distally directed contact with the proximal end of an engaging element 114.

In an exemplary embodiment, the pawl 136 is positioned at a distal end 144 of the channel 132. It will also be understood by those of skill in the art that the pawl 136 may be positioned in any of a variety of positions along the channel 132 so long as the pawl 136 is configured to engage the engaging elements 114, as described.

In an exemplary embodiment, the channel 132 includes a flat or other structure configured to be keyed to a corresponding flat or other structure of the control member 116, as will be described in further detail below. In another exemplary embodiment, a proximal face 146 of the grommet 112 is shaped (or otherwise keyed) to engage a corresponding structure of a portion of the control member 116. It will be understood by those of skill in the art, however, that the grommet 112 and the control member 116 may be keyed to one another in any of a variety of ways so long as a rotation of the control member 116 about a longitudinal axis thereof, correspondingly rotates the ligating device 102, as will be described in further detail below.

The midsection 130 extends longitudinally, distally from a wall 134 of the grommet 112 so that a longitudinal axis of the channel 132 is laterally offset from (e.g., parallel to) a longitudinal axis of the midsection 130. As will be described in further detail, upon tightening of the ligating device 102 about the target tissue, a length of the midsection 130 will extend about the target tissue, closing off a vessel thereof to prevent bleeding. Thus, a length of the midsection 130 may be selected based on a size of the target tissue structure so that it is sufficient to ligate the target tissue when tightened thereabout.

As described above, the engaging elements 114 formed along the distal section 126 of the proximal portion 108 are configured to engage the pawl 136 of the grommet 112 via, for example, a ratchet mechanism. The distal section 126 includes a plurality of engaging elements 114 formed in series therealong, with each of the engaging elements 114 being configured to engage the pawl 136 as described above so that the ligating device 102 may be tightened to a desired degree and locked at the desired degree of tightness.

In an exemplary embodiment, the engaging elements 114 may be configured as a plurality of conical or otherwise tapered elements where each of the engaging elements 114 increases in diameter from a first end 148 toward a second end 150. It is noted that when the ligating device 102 is in the straightened insertion configuration, the second end 150 is positioned proximally of the first end 148. As the distal section 126 is drawn through the channel 132 of the grommet 112, however, the second end 150 of each of the engaging elements 114 will be positioned distally of the first end 148 so that the second end 150 engages the pawl 136 once it is moved proximally therepast. Thus, the above discussion of the engaging elements 114 refers to the first ends 148 of the engaging elements 114 as distal ends and the second ends 150 are referred to as proximal ends of the engaging elements 114.

According to an exemplary embodiment, an exterior surface 151 of each of the engaging elements 114, which is angled with respect to a centrally extending longitudinal axis of the distal section 126, slides proximally along the pawl 136, deforming or otherwise moving the pawl 136 from the first position toward the second position to permit the engaging element 114 to be moved proximally therealong. Once the second end 150 has moved proximally past the pawl 136, the pawl 136 reverts to its biased first configuration, “clicking” into place immediately distal of the second end 150 so that the second end 150 engages the engaging element 114 and prevents the engaging element 114 from being moved in a distal direction relative thereto.

While each of the engaging elements 114 is shown and described as having a substantially conical shape, it will be understood by those of skill in the art that the engaging elements 114 may have any of a variety of configurations so long as the engaging elements 114 are configured to engage the pawl 136, as described herein. In another embodiment, for example, the distal section 126 may include a plurality of ratchet teeth or other similarly configured structures extending therealong.

The control member 116 extends longitudinally from a proximal end accessible to an operator (e.g., physician) of the system to a distal end 154 including the grasper 118. The control member 116 includes a shoulder 156 and a distal portion 158 extending distally therefrom. The distal portion 158 of the control member 116 is sized, shaped, and configured to be received within the channel 132 of the grommet 112 while the shoulder 156 is sized, shaped and configured to abut the proximal face 146 of the grommet 112, as shown in FIGS. 3-4. The shoulder 156 may have an increased diameter relative to the distal portion 158. In an exemplary embodiment, the shoulder 156 abuts the proximal face 146 of the grommet 112 so that distal motion of the control member 116 relative to the catheter 110 of the delivery device 104 will correspondingly move the ligating device 102 in a distal direction relative to the catheter 110. In addition, the shoulder 156 may also be configured to act a stop, which defines a distal-most position of the control member 116 relative to the ligating device 102.

A length of the distal portion 158 may be selected so that, when the ligating device 102 is in the extended configuration with the ligating device 102 looped about and encircling the target tissue, the grasper 118 at the distal end 154 may grasp the distal end 120 of the ligating device 102. The grasper 118 may have any of a number of configurations so long as the grasper 118 is configured to grasp or otherwise engage the distal end 120. In an exemplary embodiment, the grasper 118 is configured as a hook configured to be hooked over the loop 124 at the distal end 120. In another embodiment, the grasper 118 is configured as forceps configured to grip a portion of the distal end 120 of the ligating device between jaws thereof.

The delivery device 104 includes the catheter 110 and a handle assembly 160. The catheter 110 extends longitudinally from a proximal end 162 to a distal end 164 and includes a lumen 166 extending longitudinally therethrough. The catheter 110 may be sized, shaped, and configured to be inserted through, for example, a working channel of an endoscope to a target area within a patient body. The lumen 166 is sized, shaped, and configured to slidably receive the control member 116 and the ligating device 102 therein. The handle assembly 160 is positioned at the proximal end 162 of the catheter 110 so that the handle assembly 160 is connected to the proximal end of the control member 116. In an exemplary embodiment, the handle assembly 160 includes a handle member 168 configured to be gripped via the operator of the system 100, a first knob 170 configured to control a longitudinal movement of the control member 116 relative to the catheter 110, and a second knob 172 configured to control an axial motion of the control member 116 relative to the catheter 110 (i.e., rotation of the control member 116 about a longitudinal axis thereof).

According to an exemplary embodiment, the first knob 170 is connected to the proximal end of the control member 116 so that a rotation of the first knob 170 in a first direction moves the control member 116 distally relative to the catheter 110 and a rotation of the first knob in a second direction opposite the first direction moves the control member 116 proximally relative to the catheter 110. Thus, the first knob 170 is moved in the first direction to move the ligating device 102 from the insertion configuration toward the extended configuration. When the first knob 170 is moved in the second direction, the control member 116, upon grasping of the distal end 120 of the ligating device 102, is drawn proximally relative to the catheter 110 to tighten the ligating device 102 about the target tissue. In an exemplary embodiment, the first knob 170 is configured as a thumb wheel connected to the handle member 168 so that the operator may comfortably grip the handle member while rotating the first knob 170 with their thumb.

The second knob 172 may be connected to the proximal end of the control member 116 so that rotation thereof correspondingly rotates the control member 116 relative to the catheter 110, about the longitudinal axis thereof. In an exemplary embodiment, the second knob may be positioned between the handle member 168 and the proximal end 162 of the catheter 110, coaxially with the catheter 110. According to an exemplary embodiment, an interior of the second knob 172, which engages the proximal end of the control member 116 is keyed to a portion of the control member 116 received therein so that a rotation of the second knob 172 translates to a corresponding rotation of the control member 116. Thus, the rotation of the second knob 172 in a first direction relative to the catheter 110 correspondingly rotates the control member 116, and thereby the ligating device 102, in the first direction. Rotation of the second knob 172 in a second direction relative to the catheter 110, opposite the first direction, correspondingly rotates the control member 116, and thereby the ligating device 102, in the second direction.

It will be understood by those of skill in the art, however, that the handle assembly 160 may have any of a variety of configurations and any of variety of actuating mechanisms so long as the control member 116 so long as the handle member 160/actuating mechanisms facilitate movement of the control member 116, as described herein.

According to an exemplary method utilizing the system 100, a ligating device 102, housed within the delivery device 104 in the insertion configuration, may be inserted to a target area within a patient's body via, for example, a working channel of a flexible endoscope or any other suitable insertion device. As described above, in the insertion configuration, the ligating device 102 is housed within the channel of the catheter 110 in a substantially straightened configuration. The catheter 110 of the delivery device 104 may be inserted to the target area so that the distal end 164 thereof is proximate a target portion of tissue to be treated.

The ligating device 102 may then be moved from the insertion configuration toward the extended configuration by moving the ligating device 102 distally out of the catheter 110. In an exemplary embodiment, the ligating device 102 may be moved toward the extended configuration by moving the control member 116 distally with respect to the catheter 110 via, for example, the first knob 170 of the handle assembly 160. As described above, the control member 116 includes a shoulder 156 which abuts, for example, the proximal face 146 of the grommet 112 so that moving the control member 116 distally relative to the catheter 110 correspondingly moves the ligating device 102 distally with respect to the catheter 110.

As the ligating device 102 is moved distally out of the catheter 110, from the insertion configuration toward the extended configuration, the distal portion 106 of the ligating device 102 is freed to revert toward its predefined curved shape. The ligating device 102 may also be rotated about the longitudinal axis thereof, relative to the catheter 110, via, for example, the second knob 172 of the handle assembly 160, so that the ligating device 102 is in a desired orientation facilitating its placement over and around the target portion of tissue—e.g., the base of the pedicle of a polyp—as desired. Thus, as the ligating device 102 is moved out of the catheter 110, the distal portion 106 extends about the target tissue to be looped about and/or encircle the target tissue. Once the ligating device 102 has been positioned to fully encircle the target portion of tissue, the grasper 118 at the distal end 154 of the control member 116 is utilized to grasp the distal end 120 of the ligating device. In an exemplary embodiment, as described above, the grasper 118 is configured as a hook which hooks the loop 124 at the distal end 120.

Upon grasping of the distal end 120 of the ligating device 102, the control member 116 is moved proximally with respect to the catheter 110 to draw the distal end proximally through the channel 132 of the grommet 112, as shown in FIG. 7, tightening the ligating device 102 about the target portion of tissue. The operator may continue to move the distal end 120 proximally through the lumen 166 of the catheter 110 until the engaging elements 114 engage the pawl 136 within the channel 132 of the grommet 112, and the ligating device 102 has been tightened about the target tissue, as desired.

Once the ligating device 102 has been tightened as desired and the ligating device 102 is maintained in this tightened configuration via engagement of a corresponding one of the engaging elements 114 with the pawl 136, the ligating device 102 is finally deployed by releasing the distal end 120 from the grasper 118 so that the delivery device 104, including the control member 116, may be withdrawn from the patient's body, leaving just the deployed ligating device 102, as shown in FIG. 8 in position ligating the target portion of tissue. In an exemplary embodiment, the grasper 118 releases the distal end 120 by releasing a tension therealong. For example, the control member 116 may be moved slightly distally with respect to the catheter 110 so that the hook disengages the loop 124 at the distal end 120 of the ligating device 102. The control member 116 may then be rotated, orienting the hook away from the loop 124 so that the control member 116 may be proximally withdrawn without engaging the distal end 120.

It will be apparent to those skilled in the art that various modifications may be made in the present disclosure, without departing from the scope of the disclosure. Furthermore, those skilled in the art will understand that the features of any of the various embodiments may be combined in any manner that is not inconsistent with the description and/or the functionality of the embodiments.

1-15. (canceled)

16. A system for ligating tissue, comprising:
a delivery device including a catheter extending longitudinally from a proximal end to a distal end and defining a lumen extending therethrough; and
a ligating device slidably received within the lumen of the catheter and including a proximal portion configured as a fastener and a distal portion extending along a predefined curve shape, the ligating device configured to be moved from an insertion configuration, in which the ligating device is housed within the lumen of the catheter such that the ligating device is held in a straightened configuration, to an extended configuration, in which the ligating device is moved distally out of the catheter so that the distal portion reverts toward the predefined curve shape to extend about a target tissue, the proximal portion including a grommet at a proximal end thereof, the grommet including a channel extending therethrough so that when a distal end of the ligating device is drawn proximally through the channel of the grommet, the ligating device is configured to be tightened about the target tissue.

17. The system of claim 16, further comprising:
a control member slidably received within the catheter and extending longitudinally from a proximal end to a distal end including a grasper configured to grasp the distal end of the ligating device, the control member including a shoulder and a distal portion extending distally therefrom to be received within the channel of the grommet, the shoulder being sized, shaped and configured to abut a portion of the ligating device such that a distal movement of the control member relative to the catheter correspondingly moves the ligating device distally relative to the catheter.

18. The system of claim 17, wherein the grasper is configured as a hook.

19. The system of claim 17, wherein the distal end of the ligating device includes a loop configured to be grasped via the grasper of the control member.

20. The system of claim 16, wherein the proximal portion is defined via a proximal section including the grommet and a distal section including engaging elements, each of the engaging elements configured to engage a portion of the grommet as the engaging elements are drawn proximally through the channel of the grommet to tighten the ligating device about the target tissue.

21. The system of claim 20, wherein the engaging element and the channel of the grommet engage one another via a ratchet mechanism.

22. The system of claim 20, wherein the proximal section and the distal section are connected to one another via a midsection, the midsection extending distally from a wall of the grommet so that a longitudinal axis of the midsection is offset from a longitudinal axis of the channel of the grommet.

23. The system of claim 16, wherein the distal portion of the ligating device is formed of a Nitinol wire.

24. The system of claim 16, wherein the proximal portion of the ligating device is formed of a polymer material.

25. The system of claim 17, wherein the delivery device further includes a handle assembly connected to the proximal end of the catheter and to the proximal end of the control member, the handle assembly including a first actuator for controlling a longitudinal movement of the control member relative to the catheter and a second actuator for controlling an axial motion of the control member relative to the catheter.

26. A ligating system, comprising:
a catheter extending from a proximal end to a distal end and including a lumen extending therethrough;
a ligating device extending along a longitudinal axis from a proximal end including a grommet to a distal end, a distal portion of the ligating device formed of a wire extending along a predefined curve shape, the ligating device configured to be moved from an insertion configuration, in which the ligating device is housed within the lumen of the catheter such that the distal portion is held in a straightened configuration, to an extended configuration, in which the ligating device is moved distally out of the catheter so that the distal portion reverts toward the predefined curve shape to extend about a target tissue; and
a control member slidably received within the lumen of the catheter and extending longitudinally from a proximal end to a distal end including a grasper, the control member including a shoulder and a distal portion extending therefrom, the shoulder configured to abut a portion of the ligating device so that a longitudinal movement of the control member relative to the catheter correspondingly moves the ligating device longitudinally relative to the catheter, the distal portion of the control member received within a channel of the grommet so that the grasper extends distally therefrom to grasp the distal end of the ligating device and draw the distal end of the ligating device proximally through the channel of the grommet to tighten the ligating device about the tighten the ligating device about the target tissue.

27. The system of claim 26, wherein the distal end of the ligating device includes a loop configured to be engaged via a hook of the grasper.

28. The system of claim 26, wherein the ligating device includes a proximal portion from which the distal portion extends, the proximal portion defined via a proximal section including the grommet and a distal section including engaging elements, each of the engaging elements configured to engage a portion of the grommet via a ratchet mechanism as the engaging elements are drawn proximally through the channel of the grommet to tighten and fix the ligating device about the target tissue.

29. The system of claim 28, wherein the proximal section and the distal section are connected to one another via a midsection, the midsection extending distally from a wall of the grommet so that a longitudinal axis of the midsection is offset from a longitudinal axis of the channel of the grommet.

30. The system of claim 28, wherein the distal portion of the ligating device is formed of a Nitinol wire and the proximal portion of the ligating device is formed of a polymer material.

31. A method for ligating tissue, comprising:
advancing a ligating device to a target area within a patient body, in an insertion configuration in which the ligating device is housed within a catheter of a delivery device;
moving the ligating device from the insertion configuration toward an extended configuration in which the ligating device is moved distally out of the catheter so that a distal portion thereof reverts toward a predefined curve shape to extend about a target tissue; and
drawing a distal end of the ligating device through a channel of a grommet at a proximal end of the ligating device to tighten a loop of the ligating device formed about the target tissue.

32. The method of claim 16, further comprising:
drawing the distal end of the ligating device further proximally relative to the grommet until a desired level of tightness is achieved and engaging structures along the ligating device engage a portion of the grommet to hold the ligating device in the desired tightened configuration to ligate the target tissue.

33. The method of claim 31, further comprising:
rotating the ligating device relative to the catheter to position the ligating device in a desired orientation relative to the target tissue so that the target tissue extends about the target tissue as it is moved toward the extended configuration.

34. The method of claim 31, wherein the ligating device is moved from the insertion configuration toward the extending configuration via a control member slidably received within the catheter, the control member including a shoulder sized, shaped and configured to abut a portion of the ligating device such that a distal movement of the control member relative to the catheter correspondingly moves the ligating device distally relative to the catheter.

35. The method of claim 34, wherein a grasper at a distal end of the control member grasps the distal end of the ligating device to draw the distal end proximally through the channel of the grommet.
